(19) United States
(12) Patent Application Publication
(10) Pub. No.: US 2012/0191188 A1
(43) Pub. Date: Jul. 26, 2012

(54) Spinal implant with bone engaging projections

(76) Inventor: Meng-Feng Huang, Changhua (TW)

(21) Appl. No.: 13/009,851

(22) Filed: Jan. 20, 2011

(51) Int. Cl. A61F 2/44

(52) U.S. Cl. 623/17.11

(57) ABSTRACT

A spinal implant in one embodiment includes an implant for insertion between two opposite spaced vertebrae of a spine, comprising a body having a substantially rectangular cross section and comprising a toothed top retaining member, a toothed bottom retaining member, and a peripheral surface; a three-dimensional matrix structure formed in the body and on the peripheral surface as support; and a plurality of holes formed through at least one of three directions of the three-dimensional matrix structure.

### BACKGROUND OF THE INVENTION

1. Field of Invention

The invention relates to surgical procedures for stabilizing the spine and more particularly to an improved implant having bone engaging projections for use in such procedure.

2. Description of Related Art

In human anatomy, the vertebral column (backbone or spine) is a column usually consisting of 24 articulating vertebrae (including 7 vertebrae in cervical region, 12 vertebrae in thoracic region, and 5 vertebrae in lumbar region) and 9 fused vertebrae in the sacrum and the coccyx. It is situated in the dorsal aspect of the torso, separated by intervertebral discs. It houses and protects the spinal cord in its spinal canal.

Intervertebral discs lie between adjacent vertebrae in the spine. Each intervertebral disc forms a cartilaginous joint to allow slight movement of the vertebrae, and acts as a ligament to hold the vertebrae together. An intervertebral disc consists of an outer annulus fibrosus surrounding the inner nucleus pulposus. The annulus fibrosus consists of several layers of fibrocartilage. The strong annular fibers contain the nucleus pulposus and distribute pressure evenly across the disc. The nucleus pulposus contains loose fibers suspended in a mucoprotein gel with the consistency of jelly. The nucleus of the intervertebral disc acts as a shock absorber, absorbing the impact of the body's daily activities and keeping the two vertebrae separated.

Chronic low back pain is a perplexing problem facing the field of orthopedic surgery. Low back pain can be avoided by preventing relative motion between spinal vertebrae (commonly known as intervertebral stabilization). To abate low back pain, stabilization is directed to stabilizing contiguous vertebrae in the lumbar region of the spine. Surgical techniques seek to rigidly join vertebrae which are separated by a degenerated disc. One typical technique is to partially remove a degenerated disc and to insert a bone graft into the void formed by the removed disc. Spinal implants are also employed and are either acting along or in combination with bone fragments to replace the use of bone grafts.

However, improvements of spinal implant are still desired in order to enhance patient safety and the probability of a satisfactory recovery.

### SUMMARY OF THE INVENTION

It is therefore one object of the invention to provide an implant for insertion between two opposite spaced vertebrae of a spine, comprising a body having a substantially rectangular cross section and comprising a toothed top retaining member, a toothed bottom retaining member, and a peripheral surface; a three-dimensional matrix structure formed in the body and on the peripheral surface as support; and a plurality of holes formed through at least one of three directions of the three-dimensional matrix structure.

It is another object of the invention to provide an implant for insertion between two opposite spaced vertebrae of a spine, comprising a body having a substantially rectangular cross section and comprising a toothed top retaining member, a toothed bottom retaining member, and a peripheral surface; a three-dimensional matrix structure formed in the body and on the peripheral surface as support; a plurality of holes formed through at least one of three directions of the three-dimensional matrix structure; and a longitudinal channel formed in a central portion of the body and communicating with the holes.

The above and other objects, features and advantages of the invention will become apparent from the following detailed description taken with the accompanying drawings.

### BRIEF DESCRIPTION OF THE DRAWINGS

FIG. 1 is a perspective view of a spinal implant according to a first preferred embodiment of the invention;

FIGS. 2 and 3 are sectional views taken along line 2-2 and line 3-3 of FIG. 1 respectively;

FIG. 4 schematically depicts a fixing of the spinal implant in a bore formed between opposing vertebrae of a spine;

FIG. 5 is a perspective view of a spinal implant according to a second preferred embodiment of the invention;

FIGS. 6 and 7 are sectional views taken along line 6-6 and line 7-7 of FIG. 5 respectively;

FIG. 8 schematically depicts a fixing of the spinal implant of FIG. 5 in a bore formed between opposing vertebrae of a spine;

FIG. 9 is a perspective view of a spinal implant according to a third preferred embodiment of the invention; and

FIG. 10 is a perspective view of a spinal implant according to a fourth preferred embodiment of the invention.

### DETAILED DESCRIPTION OF THE INVENTION

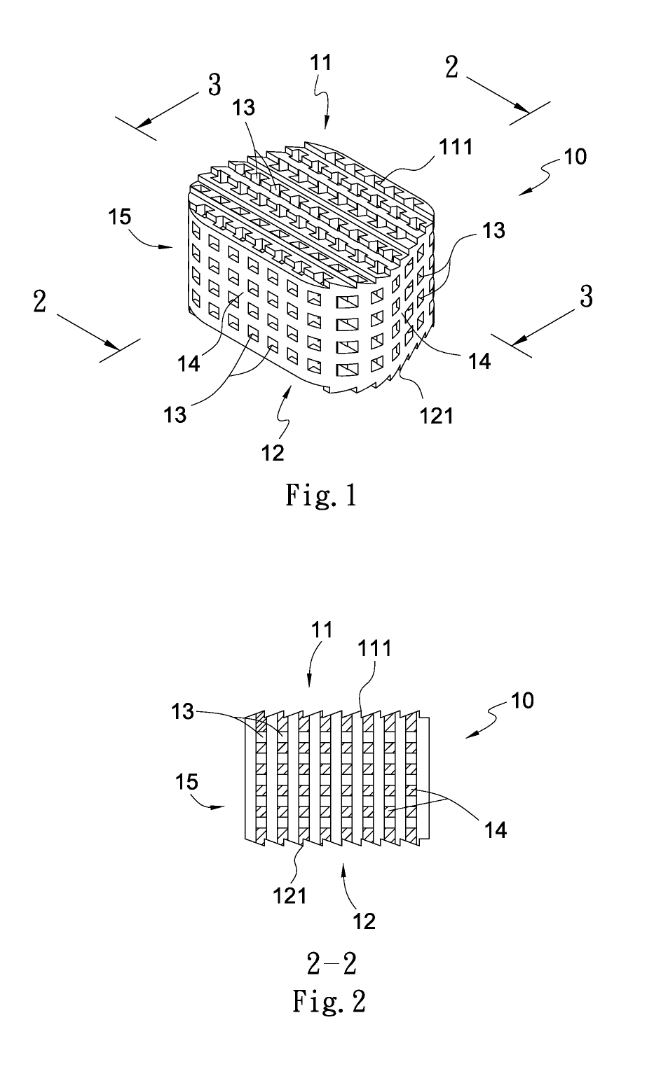

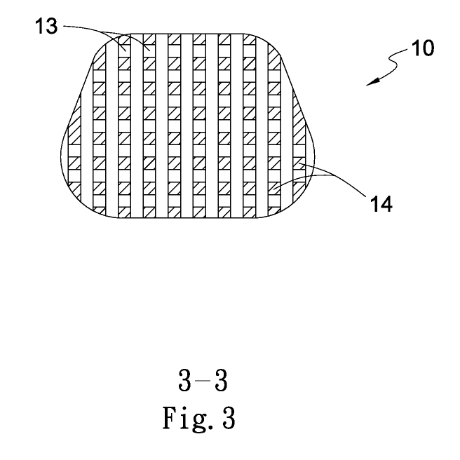

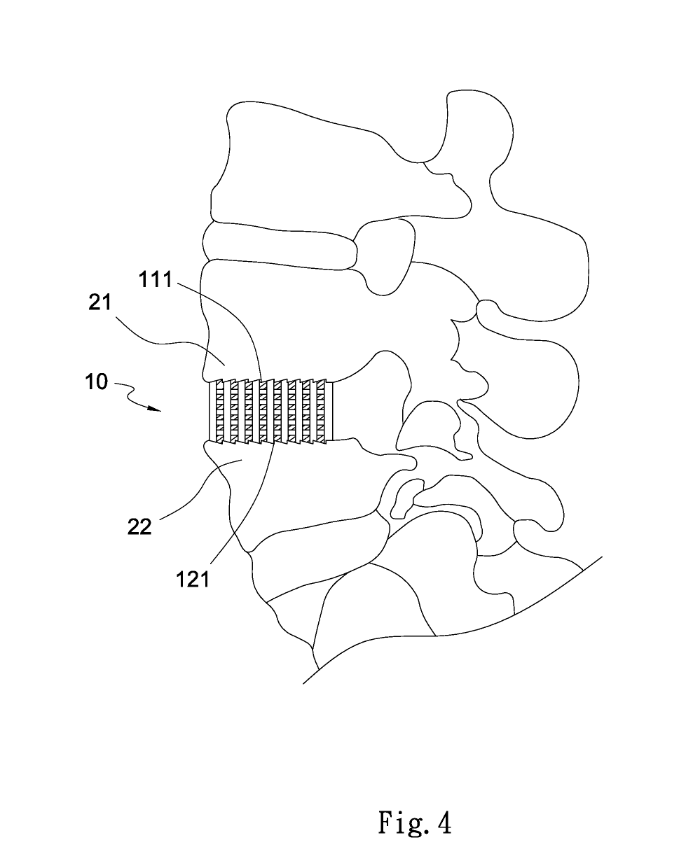

Referring to FIGS. 1 to 4, a spinal implant in accordance with a first preferred embodiment of the invention comprises the following components as discussed in detail below.

A body 10 has a substantially rectangular cross section and comprises a top surface 11, a bottom surface 12, and a peripheral surface 15 therebetween. The body 10 is formed of a composite material being sturdy and highly resistant to chemicals. The composite material may be carbon fiber or PEEK (polyetheretherketone). Alternatively, the body 10 is formed of alloy such as stainless steel, cobalt-chromium-molybdenum alloy, titanium, or titanium alloy. Still alternatively, the body 10 is formed of polymer such as UHMWPE (ultra high molecular weight polyethylene), PMMA (polymethylmethacrylate), silicon rubber, or ultra high molecular polyethylene. Still alternatively, the body 10 is formed of ceramic such as aluminum oxide, calcium phosphate tri-basic, or fiber glass.

An upper retaining member 111 is formed on the top surface 11 and shaped as a plurality of rows of bone engaging projections (i.e., teeth). A lower retaining member 121 is formed on the bottom surface 12 and shaped as a plurality of rows of bone engaging projections (i.e., teeth). A three-dimensional matrix structure 14 is formed in the body 10 and on the peripheral surface 15 as support. The three-dimensional matrix structure 14 can provide mechanical properties such as enhanced resistance to pressure, enhanced resistance to stress, and enhanced resistance to tension to the body 10. A plurality of holes 13 are formed through each of three directions of the three-dimensional matrix structure 14. The holes 13 occupy about 1% to 90% of the volume of the body 10 depending upon engineering choice of design. Each hole 13 has a bore of about 150 μm to 1,000 μm.

As shown in FIG. 4 specifically, a physician may insert the body 10 into a bore formed between an upper vertebra 21 and a lower vertebra 22 of a spine. The bone engaging projections of the upper retaining member 111 and the bone engaging projections of the lower retaining member 121 thus grasped the upper vertebra 21 and the lower vertebra 22 respectively (i.e., the body 10 being fastened). Moreover, an appropriate biocompatible material may filled in the voids of the holes 13 for stuffing and stabilization purposes. The biocompatible material may be calcium phosphate tri-basic (CaP) or hydroxyapattie (HA) (Ca₁₀(PO₄)₆(OH)₂).

The holes 13 allow body tissues to grow therein for the health of spinal bone. Moreover, the fastening of the upper and lower vertebrae 21, 22 and the upper and lower retaining members 111, 112 is reliable so as to rigidly join the upper and lower vertebrae 21, 22. As a result, intervertebral stabilization is carried out.

In addition, the provision of the three-dimensional matrix structure 14 can significantly increase resistance to pressure, stress, and tension to the body 10. Therefore, the body 10 is sturdy and a useful life of the spinal implant can be prolonged.

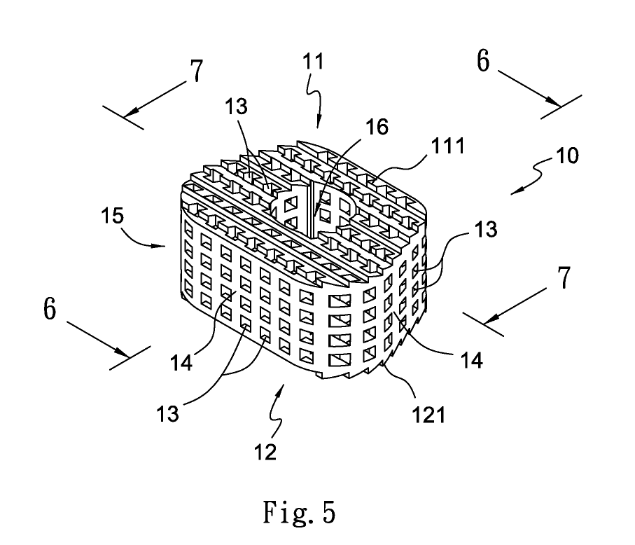

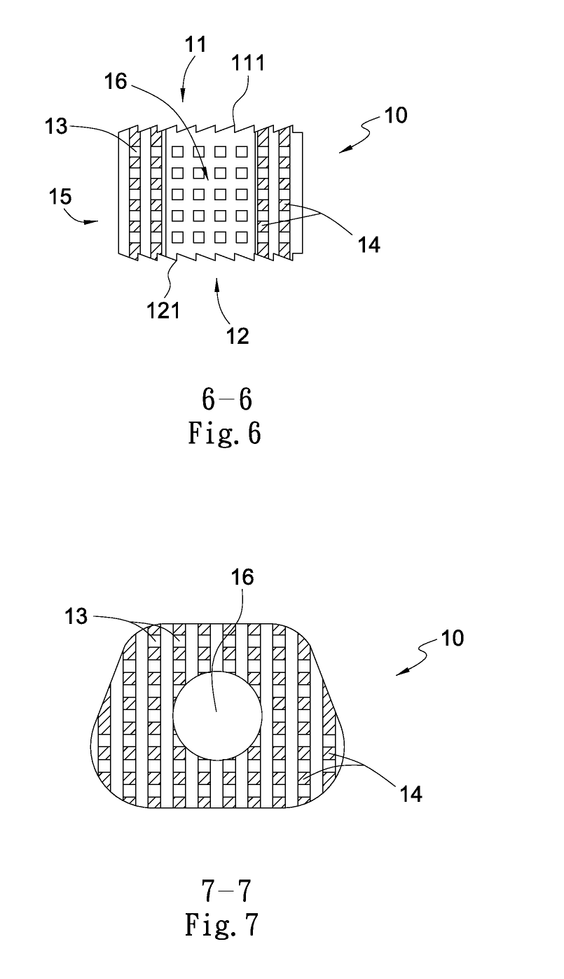

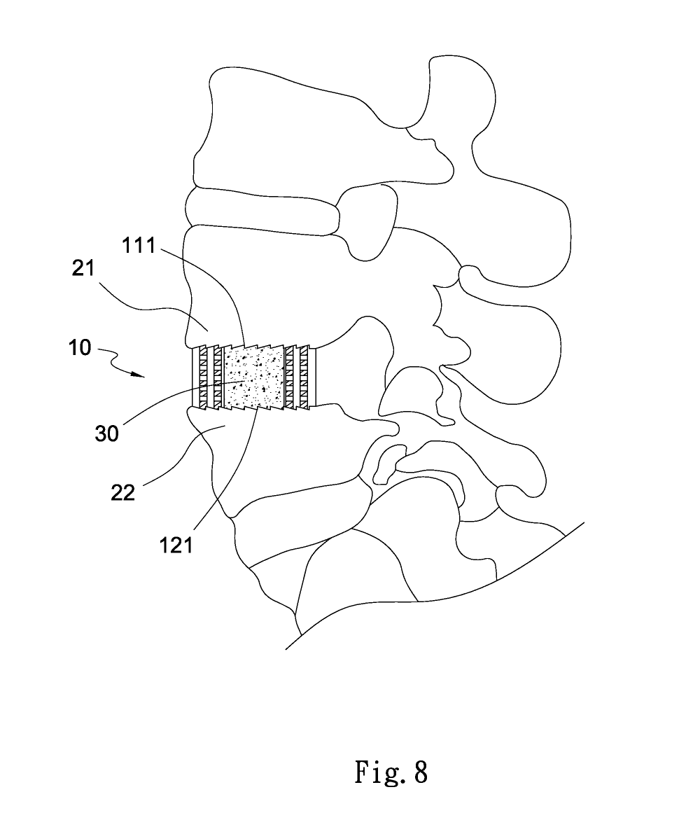

Referring to FIGS. 5 to 8, a spinal implant in accordance with a second preferred embodiment of the invention is shown. The characteristics of the second preferred embodiment are substantially the same as that of the first preferred embodiment except the following: A longitudinal channel 16 of circular section is formed in a central portion of the body 10 and communicates with the holes 13. After inserting the spinal implant between the upper and lower vertebrae 21, 22 with the upper and lower vertebrae 21, 22 and the upper and lower retaining members 111, 112 being fastened together in a surgery, bone tissues 30 may grow to fill in the channel 16. This has the benefits of carrying out intervertebral stabilization and increasing the probability of a satisfactory recovery of a patient.

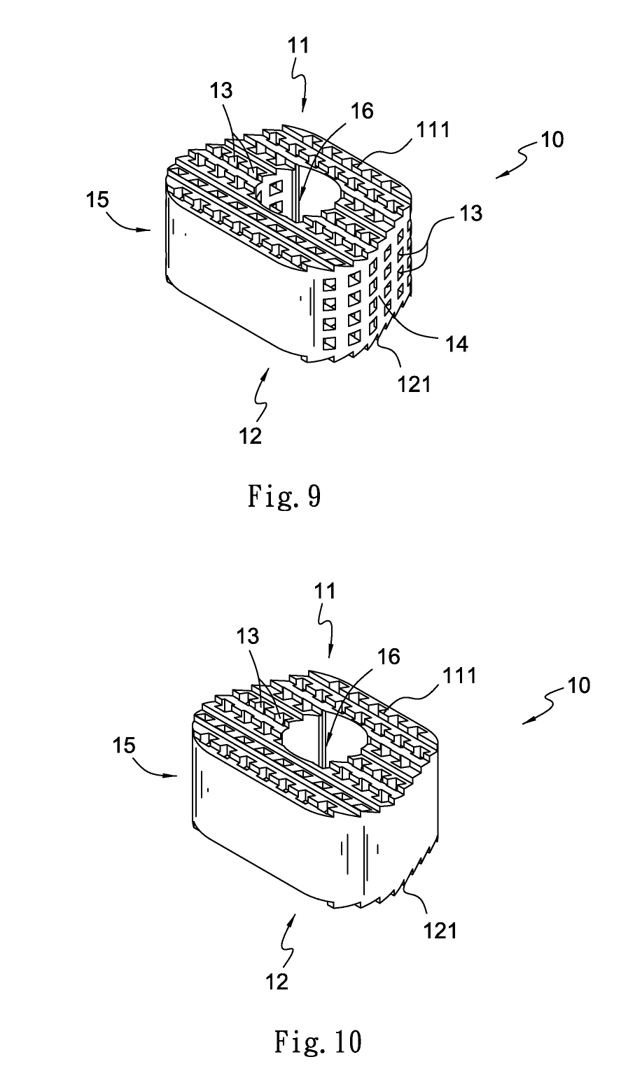

Referring to FIG. 9, a spinal implant in accordance with a third preferred embodiment of the invention is shown. The characteristics of the third preferred embodiment are substantially the same as that of the second preferred embodiment except the following: The peripheral surface 15 has two relatively smooth opposite sides, an inner surface of the channel 16 has two opposite smooth portions substantially aligned with the two opposite sides of the peripheral surface 15, and no holes 13 are formed along the direction of the opposite sides of the peripheral surface 15.

Referring to FIG. 10, a spinal implant in accordance with a fourth preferred embodiment of the invention is shown. The characteristics of the fourth preferred embodiment are substantially the same as that of the second preferred embodiment except the following: The peripheral surface 15 has all of four sides being relatively smooth, the channel 16 has a relatively smooth inner surface, and no holes 13 being formed transversely (i.e., the body 10 having no transverse holes 13).

While the invention has been described in terms of preferred embodiments, those skilled in the art will recognize that the invention can be practiced with modifications within the spirit and scope of the appended claims.

What is claimed is:

1. An implant for insertion between two opposite spaced vertebrae of a spine, comprising:
a body having a substantially rectangular cross section and comprising a toothed top retaining member, a toothed bottom retaining member, and a peripheral surface;
a three-dimensional matrix structure formed in the body and on the peripheral surface as support; and
a plurality of holes formed through at least one of three directions of the three-dimensional matrix structure.

2. The spinal implant of claim 1, wherein the holes are formed through one transverse direction of the three directions of the three-dimensional matrix structure.

3. The spinal implant of claim 1, wherein the holes are formed through two transverse directions of the three directions of the three-dimensional matrix structure.

4. The spinal implant of claim 1, wherein the holes are formed through each of the three directions of the three-dimensional matrix structure.

5. The spinal implant of claim 1, wherein the holes occupy about 1% to 90% of a volume of the body, and wherein each hole has a bore of about 150 μm to 1,000 μm.

6. An implant for insertion between two opposite spaced vertebrae of a spine, comprising:
a body having a substantially rectangular cross section and comprising a toothed top retaining member, a toothed bottom retaining member, and a peripheral surface;
a three-dimensional matrix structure formed in the body and on the peripheral surface as support;
a plurality of holes formed through at least one of three directions of the three-dimensional matrix structure; and
a longitudinal channel formed in a central portion of the body and communicating with the holes.

7. The spinal implant of claim 6, wherein the holes are formed through one transverse direction of the three directions of the three-dimensional matrix structure.

8. The spinal implant of claim 6, wherein the holes are formed through two transverse directions of the three directions of the three-dimensional matrix structure.

9. The spinal implant of claim 6, wherein the holes are formed through each of the three directions of the three-dimensional matrix structure.

10. The spinal implant of claim 6, wherein the holes occupy about 1% to 90% of a volume of the body, and wherein each hole has a bore of about 150 μm to 1,000 μm.
